(19) United States
(12) Patent Application Publication
(10) Pub. No.: US 2021/0346319 A1
(43) Pub. Date: Nov. 11, 2021

(54) Pharmaceutical Compositions of R-(+)-Propranolol in Enantiomeric Excess and Therapeutic Uses Related Thereto

(71) Applicant: Emory University, Atlanta, GA (US)

(72) Inventor: Jack Arbiser, Atlanta, GA (US)

(21) Appl. No.: 17/282,632

(22) PCT Filed: Sep. 27, 2019
(86) PCT No.: PCT/US2019/053491

§ 371 Date: Apr. 2, 2021

Related U.S. Application Data
(60) Provisional application No. 62/741,185, filed on Oct. 4, 2018.

(51) Int. Cl. A61K 31/138; A61K 31/404; A61K 31/165; A61K 31/167; A61K 39/395; A61K 31/5377; A61K 31/704; A61K 31/506; A61K 31/519; A61K 31/337; A61K 31/517; A61K 31/675; A61K 31/4184; A61K 31/4745; A61P 35/00

(52) U.S. Cl. CPC A61K 31/138; A61P 35/00; A61K 31/165; A61K 31/167; A61K 39/3955; A61K 31/5377; A61K 31/704; A61K 31/506; A61K 31/519; A61K 31/337; A61K 31/517; A61K 31/675; A61K 31/4184; A61K 31/4745; A61K 31/404

(57) ABSTRACT

In certain embodiments, this disclosure relates to methods of treating or preventing angiogenic disorders such as cancer, skin cancer, angiogenic disorders of the skin, and inflammatory disorders comprising administering an effective amount of a composition comprising (R)-(+)-1-(isopropylamino)-3-(naphthalen-1-yloxy)propan-2-ol or salt thereof in enantiomeric excess to a subject in need thereof. In certain embodiments, this disclosure contemplates compositions or pharmaceutical compositions comprising (R)-(+)-1-(isopropylamino)-3-(naphthalen-1-yloxy)propan-2-ol or salt thereof in enantiomeric excess as reported herein.

### CROSS-REFERENCE TO RELATED APPLICATIONS

This application claims the benefit of U.S. Provisional Application No. 62/741,185 filed Oct. 4, 2018. The entirety of this application is hereby incorporated by reference for all purposes.

### STATEMENT REGARDING FEDERALLY SPONSORED RESEARCH OR DEVELOPMENT

This invention was made with government support under AR047901 awarded by the National Institutes of Health. The government has certain rights in the invention.

### BACKGROUND

Angiogenesis is the growth of blood vessels from the existing vasculature. Angiogenesis is a normal and vital process in development, wound healing, and in the formation of granulation tissue. However, it is also a fundamental step in the transition of tumors from a benign to a malignant state. Angiogenesis is also involved in the formation of vascular lesions such as hemangiomas of infancy (HOI), vascular malformations, and malignant vascular tumors. Leaute-Labreze et al. report using propranolol for treating severe hemangiomas of infancy. N Engl J Med, 2008, 358(24), 2649. See also U.S. Pat. No. 8,633,181 and Russian Patent No. 2471500, which report the use of beta-blockers for treating hemangiomas.

Propranolol is traditionally used clinically for treating hypertension, atrial fibrillation, and preventing a heart attack. Pharmaceutical compositions of propranolol are a racemic mixture of two enantiomers, R(+) and S(−), e.g. Inderal® (±)-1-[(1-methylethyl)amino]-3-(1-naphthalenyloxy)-2-propanol hydrochloride, and is known to have activity as a beta-adrenergic receptor blocking agent, otherwise known as beta-blockers. As beta-blockers alter functions of the heart and circulatory system, side effects include bradycardia (slow heart rate) and hypotension (low blood pressure). Co-administration of numerous drugs with propranolol are reported to lead to clinically relevant drug interactions and changes in efficacy and/or toxicity. Thus, avoiding these side effects are desirable.

Howe et al. report optical isomers of propranolol. Nature, 1966, 210, 1336-1338.

References cited herein are not an admission of prior art.

### SUMMARY

In certain embodiments, this disclosure relates to methods of treating or preventing angiogenic disorders such as cancer, skin cancer, angiogenic disorders of the skin, and inflammatory disorders comprising administering an effective amount of a composition comprising (R)-(+)-1-(isopropylamino)-3-(naphthalen-1-yloxy)propan-2-ol or salt thereof in enantiomeric excess to a subject in need thereof. In certain embodiments, this disclosure contemplates compositions or pharmaceutical compositions comprising (R)-(+)-1-(isopropylamino)-3-(naphthalen-1-yloxy)propan-2-ol or salt thereof in enantiomeric excess as reported herein.

In certain embodiments, this disclosure relates to methods of treating or preventing hemangiomas comprising administering an effective amount of a composition or a pharmaceutical composition comprising (R)-(+)-1-(isopropylamino)-3-(naphthalen-1-yloxy)propan-2-ol or salt thereof in enantiomeric excess as reported herein to a subject in need thereof.

In certain embodiments, this disclosure relates to methods of treating or preventing skin cancer comprising administering an effective amount of a composition or a pharmaceutical composition comprising (R)-(+)-1-(isopropylamino)-3-(naphthalen-1-yloxy)propan-2-ol or salt thereof in enantiomeric excess as reported herein to a subject in need thereof.

In certain embodiments, this disclosure relates to methods of treating or preventing melanoma comprising administering an effective amount of a composition and pharmaceutical composition comprising (R)-(+)-1-(isopropylamino)-3-(naphthalen-1-yloxy)propan-2-ol or salt thereof in enantiomeric excess as reported herein to a subject in need thereof.

In certain embodiments, the subject is at risk of, exhibiting symptoms of, or diagnosed with cancer, skin cancer, and angiogenic disorders of the skin, hemangiomas, melanoma, or other disorders described herein.

In certain embodiments, compositions are administered in combination with another chemotherapy agent such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

In certain embodiments, this disclosure relates to methods of treating or preventing cancer comprising administering an effective amount of a composition or a pharmaceutical composition comprising (R)-(+)-1-(isopropylamino)-3-(naphthalen-1-yloxy)propan-2-ol or salt thereof in enantiomeric excess as reported herein to a subject in need thereof. In certain embodiments, this disclosure relates to a medicament for uses in managing diseases or conditions as reported herein. In certain embodiments, this disclosure relates to the productions of a medicament for uses in managing diseases or conditions as reported herein.

In certain embodiments, this disclosure relates to compositions comprising (R)-(+)-1-(isopropylamino)-3-(naphthalen-1-yloxy)propan-2-ol or salt thereof in greater than 55%, 75%, 85%, 95%, 98% or more enantiomeric excess. In certain embodiments, this disclosure relates to pharmaceutical compositions comprising (R)-(+)-1-(i sopropylamino)-3 -(naphthalen-1-yloxy)propan-2-ol or salt thereof in enantiomeric excess as reported herein and a pharmaceutically acceptable excipient.

In certain embodiments, the pharmaceutical composition is in the form of a tablet, pill, capsule, gel, gel capsule, or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutical composition is in solid form surrounded by an enteric coating. In certain embodiments, the enteric coatings comprises a component such as methyl acrylate-methacrylic acid copolymers, cellulose acetate phthalate (CAP), cellulose acetate succinate, hypromellose (hydroxypropyl methylcellulose), hypromellose phthalate (hydroxypropyl methyl cellulose phthalate), hypromellose acetate succinate (hydroxypropyl methyl cellulose acetate succinate), diethyl phthalate, polyvinyl acetate phthalate (PVAP), methyl methacrylate-methacrylic acid copolymers, or combinations thereof.

In certain embodiments, the pharmaceutically acceptable excipient is selected from lactose, sucrose, mannitol, triethyl citrate, dextrose, cellulose, microcrystalline cellulose, methyl cellulose, ethyl cellulose, hydroxyl propyl cellulose, hydroxypropyl methylcellulose, carboxymethylcellulose, croscarmellose sodium, polyvinyl N-pyrrolidone (crospovidone), ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, this disclosure relates to methods of treating or preventing skin disorders, both inflammatory and malignant, such as psoriasis, nonmelanoma skin cancer, actinic keratosis, atopic dermatitis/eczema, immunobullous disorders, chronic wounds, stasis dermatitis, warts, acne, seborrheic dermatitis, scleroderma, keloids, contact and irritant dermatitis, cutaneous T cell lymphoma, tuberous sclerosis, neurofibromatosis, by administering an affective amount of a composition disclosed herein to a subject in need thereof.

### BRIEF DESCRIPTION OF THE DRAWINGS

FIG. 1 shows data using propranolol isomers on Angptl4 protein expression in hemangioma endothelial cells. R-propranolol (non-beta blocker)-treated bEnd.3 cells exhibited markedly reduced Angptl4 protein expression, while S-propranolol (beta blocker)-treated cells had slightly elevated Angptl4 level. Gentian violet had much reduced Angptl4 as well as the basal expression of the loading control, GAPDH.

FIG. 2A shows representative images of immunohistochemistry with Angptl4 of the human infantile hemangioma samples. Infantile hemangioma samples express Angptl4 proteins. Angptl4 is expressed in hemangioma of infancy.

FIG. 2B shows a negative control.

FIG. 3 shows data using R-propranolol on bEnd.3 hemangioma growth in vivo. bEnd.3 cells xenografted into mice formed tumors within two weeks of inoculation, and the treatment with R-propranolol significantly reduced the tumor volume within two weeks of tumor development (p=0.014).

FIG. 4 shows data using box plots of differentially expressed RNA transcripts of bEnd.3 cells treated with R-propranolol. Seven transcripts were found to be significantly downregulated, and seventeen transcripts were upregulated. Six of the upregulated genes of interest including Egr1, APOA1, and BHMT as well as three of the downregulated genes including Faim2, Hunk, and Eno4 are included for representation.

FIG. 5A shows data indicating R- and S-propranolol induce metabolic changes towards respiration irrespective of beta blockade. R- and S-propranolol treated bend3 cells were assayed with serial injection of oligomycin, FCCP, and combination of rotenone and antimycin A to measure ATP production, maximal respiration, and nonmitochondrial respiration respectively. The spare respiratory capacity as well as proton leakage are calculated from these measurements. a) Both R- and S-propranolol treatments substantially increased maximal respiration compared to the vehicle control. R-propranolol, however, induced stronger respiration than S-propranolol.

FIG. 5B shows data indicating R- and S-propranolol increased basal respiration as well as the spare respiratory capacity.

FIG. 5C shows data indicating that while ATP production was markedly increased in cells treated in either isomers of propranolol, proton leakage was also induced.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey or domestic pet.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, fulvestrant, bicalutamide, flutamide, nilutamide, goserelin, leuprorelin, buserelin, megestrol, anastrozole, letrozole, vorozole, exemestane, finasteride, marimastat, trastuzumab, cetuximab, dasatinib, imatinib, thalidomide, azacitidine, azathioprine, capecitabine, chlorambucil, cyclophosphamide, cytarabine, daunorubicin, doxifluridine, epothilone D, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (Off); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

### Propranolol exhibits activity against hemangiomas independent of beta blockade

Propranolol is a beta blocker that consists of a racemic mixture of R and S stereoisomers. A fortuitous clinical observation was made in an infant who received propranolol for cardiac disease, and regression of a hemangioma of infancy was noted. See Leaute-Labreze et al. entitled “Propranolol for severe hemangiomas of infancy.” The New England journal of medicine 358, 2649-2651, 2008. This has led to the use of propranolol for the treatment of large and life-threatening hemangiomas of infancy. Infants receiving propranolol require monitoring to ensure that they do not suffer from side effects related to beta blockade. The exact mechanism of activity of propranolol in hemangioma of infancy was unknown.

Experiments were performed wherein hemangioma stem cells were treated with both beta blockade active (S-propranolol) and inactive (R-propranolol) as well as the angiogenesis inhibitor gentian violet. Genes profiles were searched during treatment. Among the genes commonly downregulated, Angiopoietin like 4 (Angptl4) was prominent. Propranolol isomers downregulated Angptl4 in endothelial cells, with greater downregulation of Angptl4 using the beta blockade inactive R-propranolol. Angptl4 is present in human hemangiomas of infancy. Finally, R-propranolol inhibited the growth of bEnd.3 hemangioma cells in vivo. The implications of this is that hemangioma growth can be blocked without the side effects of beta blockade. Thus, in certain embodiments, this disclosure contemplates using R-propranolol for hemangiomas of infancy and other angiogenic and inflammatory diseases and conditions.

Hemangiomas of infancy are the most common tumor of childhood and have not consistently been associated with a specific mutation, despite being clonal. Signaling abnormalities have been described in hemangiomas of infancy, including Glut-1 expression, cytoplasmic WT-1 expression, and elevated levels of NADPH oxidase. While most hemangiomas do not require treatment, a significant subset of hemangiomas cause significant and even life-threatening consequences, including compression of the trachea, ocular damage, and disfigurement. Hemangiomas are also associated with PHAGE syndrome, in which hemangiomas are associated with other abnormalities, including posterior fossa brain malformations, and cardiac abnormalities. However, treatment of hemangiomas with propranolol is not risk free, in that propranolol may cause bradycardia, hypotension, and hypoglycemia as a consequence of beta blockade.

Experiments were performed to determine whether propranolol works through beta blockade independent mechanisms. Commercial propranolol is a mixture of S-propranolol (beta blocker) and R-propranolol (non-beta blocker). The same is true for other commercially available beta blockers, which are synthesized as aryl ethers of epichlorhydrin which are then reacted with a primary amine, leading to an optically active center which is sold as a racemic mixture, based on the assumption that the R-isomer is biologically inactive.

Purified isomers of propranolol were used to assess biological activity. Hemangioma stem cells were treated with R- and S-propranolol as well as the angiogenesis inhibitor gentian violet, which was used for the treatment of localized small ulcerated hemangiomas. The gene that was most coordinately regulated by this treatment is Angptl4, which is downregulated by all three treatments on gene array. The expression of Angptl4 protein was examined. The R-isomer (non-beta blocker) suppressed Angptl4, as did gentian violet, but the beta blocker S isomer had no effect. Using immunohistochemistry, Angptl4 was identified in authentic hemangioma of infancy.

Because R-propranolol appeared to have greater activity, and S-propranolol has a low LD₅₀ in mice due to beta blockade. The ability of R-propranolol to block the growth of bEnd.3 hemangioma in vivo was assessed because it is a useful preclinical model that recapitulates many of the signaling abnormalities of hemangioma of infancy. The bEnd.3 cells are useful for in vivo studies as human hemangioma endothelia do not grow robustly in mice. R-propranolol caused significant inhibition of tumor growth. RNAseq analysis was performed using treated vs vehicle tumors. The gene that was most upregulated by R-propranolol treatment was betaine homocysteine methyl transferase (BHMT). This enzyme converts toxic homocysteine into methionine using betaine as a methyl donor. KEGG analysis showed that choline metabolism was highly affected. Egr-1, an AP-1 subunit with tumor suppressive activity, was also elevated by R-propranolol treatment. APOA1 (high density lipoprotein), which protects against vascular instability was also induced by R-propranolol. N4RA1 (Nur77) is also upregulated by propranolol. N4RA1 is associated with tumor suppression in hepatocellular carcinoma by suppressing glycolysis.

Hemangioma of infancy is a glycolytic tumor. The effect of propranolol isoforms on metabolism in bEnd.3 hemangioma cells were examined based on PET uptake and overexpression of the glucose transporter GLUT1. Treatment with both isomers of propranolol stimulated respiration upon Seahorse analysis. This data indicates propranolol induces metabolic changes towards respiration independent of beta blockade.

### Methods of Use

Experiments indicates that propranolol (a mixture of enantiomers) coordinately downregulates VEGF/Angplt4. The isomer of propranolol that does not have beta-blocker activity (R-propranolol) is more active against Angptl4 than the beta-blocker (S-propranolol). Thus, compositions enriched with R-propranolol isomer are contemplated to be useful as a general angiogenesis inhibitor with benefits for treating or preventing tumors and inflammatory conditions.

In certain embodiments, disclosure contemplates method of treating or preventing vascular lesions and vascular malformations using compositions disclosed herein. Vascular malformations include congenital vascular anomalies of veins, lymph vessels, or arteries. Lesions may remain stable through life of patient but can cause significant morbidity and mortality through brain involvement, limb overgrowth, and lymphatic malformations. Malignant vascular tumors include several forms of malignant tumors such as hemangioendothelioma, angiosarcoma and Kasabach-Merritt syndrome.

Several classes of vascular lesions exist in humans. The most common class is hemangiomas of infancy. Hemangiomas are birthmarks that most commonly appear as a rubbery, bright red nodule of extra blood vessels in the skin. Hemangiomas typically grow during the first year of life and then recedes over time. However, sometimes treatment of a hemangioma is initiated if the nodule interferes with vision or breathing. Hemangiomas exhibit a distinct “life cycle” of rapid proliferation, involution, and finally replacement with connective tissue. Another class of vascular lesions are vascular malformations. Vascular malformations include lesions that remain stable through the life of the patient, but can cause significant morbidity and mortality through brain involvement (Sturge-Weber syndrome), limb overgrowth, and lymphatic malformations (lymphangiomas). Vascular and lymphatic malformations are typically caused by mutations in endothelial cells. These include GNAQ in Sturge-Weber syndrome, Tie2/TEK somatic mutations in venous malformations, PIK3CA activating mutations in sporadic venous malformations. Another class of vascular lesions are malignant vascular tumors. These tumors range from the low-grade hemangioendothelioma, Kasabach-Merritt syndrome, to high-grade angiosarcoma.

In certain embodiments, this disclosure contemplates the treatment for hemangiomas of infancy without the side effects of beta blockade in infants (hypoglycemia, bradycardia, etc.). Infants with PHAGE syndrome show posterior fossa anomalies (a structural brain abnormality), hemangioma (growth of blood vessels) appearing on the skin, arterial abnormalities, cardiac (heart) abnormalities, and abnormalities of the eye, have a high risk of stroke because of anomalous blood vessels of the brain. Thus, lowering blood pressure in these patients with beta-blockers may be hazardous.

It is also contemplated that systemic angiogenesis inhibitors may be beneficial for the prevention and treatment of other angiogenic disorders of the skin, including melanoma and non-melanoma skin cancer.

Propranolol downregulates VEGF levels but not Angiopoietin-2 (Ang2) levels in infants with hemangiomas. Propranolol given to patients is a racemic mixture, containing the beta blocking active isomer (S-propranolol) and beta blocking inactive isomer (R-propranolol). Experiments, reported herein indicate that the R-propranolol is the more active isoform in mediating hemangioma regression.

In certain embodiments, this disclosure relates to methods of treating or preventing skin disorders, both inflammatory and malignant, such as psoriasis, nonmelanoma skin cancer, actinic keratosis, atopic dermatitis/eczema, immunobullous disorders, chronic wounds, stasis dermatitis, warts, acne, seborrheic dermatitis, scleroderma, keloids, contact and irritant dermatitis, cutaneous T cell lymphoma, tuberous sclerosis, neurofibromatosis, by administering an affective amount of a composition disclosed herein to a subject in need thereof. In certain embodiments, the subject is at risk of, exhibiting symptoms of, suspected of, or diagnosed with skin disorders, both inflammatory and malignant, such as psoriasis, nonmelanoma skin cancer, actinic keratosis, atopic dermatitis/eczema, immunobullous disorders, chronic wounds, stasis dermatitis, warts, acne, seborrheic dermatitis, scleroderma, keloids, contact and irritant dermatitis, cutaneous T cell lymphoma, tuberous sclerosis, or neurofibromatosis.

In certain embodiments, the disclosure relates to a method of treating or preventing cancer comprising administering an effective amount of a pharmaceutical composition comprising (R)-(+)-1-(i sopropylamino)-3-(naphthalen-1-yloxy)propan-2-ol or salt thereof in enantiomeric excess as reported herein to a subject in need thereof. In certain embodiments, the subject is diagnosed with or at risk of cancer. In certain embodiments, the compound is administered in combination with a second therapeutic agent. In certain embodiments, the cancer is selected from bladder cancer, lung cancer, breast cancer, melanoma, colon and rectal cancer, non-Hodgkin lymphoma, Burkett lymphoma, endometrial cancer, pancreatic cancer, kidney cancer, prostate cancer, leukemia, thyroid cancer, and brain cancer.

In certain embodiment, this disclosure relates to methods for the treatment a subject at risk of, exhibiting symptoms of, suspected of, or diagnosed with a cancer or neoplasm selected from skin cancer, melanoma, Barret's adenocarcinoma; biliary tract carcinomas; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system), colorectal cancer, including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; and thyroid cancers.

In certain embodiments, the subject is diagnosed with cancer or hematological malignancy. In certain embodiments, the hematological malignancy is multiple myeloma, leukemia, or lymphoma. In certain embodiments, the hematological malignancy is acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), Hodgkin's lymphomas, and non-Hodgkin's lymphomas such as Burkitt lymphoma, B-cell lymphoma.

The compounds disclosed herein can be used alone in the treatment of each of the foregoing conditions or can be used to provide additive or potentially synergistic effects with certain existing chemotherapies, radiation, biological or immunotherapeutics (including monoclonal antibodies) and vaccines. Compositions and pharmaceutical compositions comprising (R)-(+)-1-(isopropylamino)-3-(naphthalen-1-yloxy)propan-2-ol or salt thereof in enantiomeric excess as reported herein may be useful for restoring effectiveness of certain existing chemotherapies and radiation and or increasing sensitivity to certain existing chemotherapies and/or radiation.

In certain embodiments, this disclosure provides a method for treating a susceptible neoplasm in a mammal in need thereof comprising the steps of: (a) analyzing a sample from said neoplasm to determine whether an activating mutation is present in the coding sequence for BRAF in cells of said neoplasm; (b) selecting a mammal having a neoplasm with an activating mutation in the coding sequence for BRAF; and (c) administering a therapeutically effective amount of a pharmaceutical composition comprising (R)-(+)-1-(isopropylamino)-3 -(naphthalen-1-yloxy)propan-2-ol or salt thereof in enantiomeric excess as reported herein to the mammal selected in step (b).

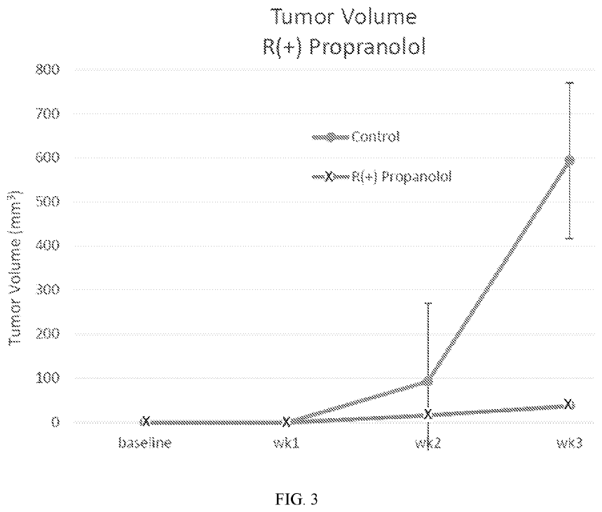

In certain embodiments, the activating mutation present in the coding sequence for BRAF results in a BRAF having an amino acid substitution selected from the group consisting of R4621, I463S, G464V, G464E, G466A, G466E, G466V, G469A, G469E, D594V, F595L, G596R, L597V, L597R, T5991, V600E, V600D, V600K, V600R, T119S, and K601E. See, for example, FIG. 2 of Halilovic and Solvit (2008) Current Opinion in Pharmacology 8:419-26.

In certain embodiments, this disclosure relates to a method for treating a susceptible neoplasm in a mammal in need thereof comprising the steps of: (a) analyzing a sample from said neoplasm to determine whether a mutation encoding a V600E, V600D or V600R amino acid substitution is present in the coding sequence for BRAF in cells of said neoplasm; (b) selecting a mammal having a neoplasm with a mutation encoding the V600E, V600D or V600R amino acid substitution in BRAF; and (c) administering a therapeutically effective amount of a pharmaceutical composition comprising (R)-(+)-1-(isopropylamino)-3-(naphthalen-1-yloxy)propan-2-ol or salt thereof in enantiomeric excess as reported herein to the mammal selected in step (b).

The V600E amino acid substitution in BRAF is described, for example, in Kumar et al. (2004) J Invest Dermatol. 122(2):342-8. This mutation commonly results from a T1799A mutation in the coding sequence for human BRAF. Accordingly, in one embodiment of the present disclosure, the step of analyzing a sample from said neoplasm to determine whether a mutation encoding a V600E amino acid substitution is present in the coding sequence for BRAF is performed by determining whether the coding sequence for BRAF in cells of the neoplasm contains the T1799A mutation.

In one preferred embodiment, the neoplasm is melanoma.

In particular embodiments, the neoplasm is selected from breast cancer, cholangiocarcinoma, colorectal cancer, melanoma, non-small cell lung cancer, ovarian cancer, and thyroid cancer.

In certain embodiments, the subject is a mammal such as a human.

Abdelmaksoud et al. report classic and HIV-related Kaposi sarcoma treated with topical timolol gel. J Am Acad Dermatol. 2017, 76(1):153-155. In certain embodiments, the R-propranolol isomer is administered in combination with another chemotherapy agent and/or beta-blocker such as timolol, carvedilol, metoprolol, atenolol, acebutolol, and combinations thereof.

Kolb et al. report obesity-associated inflammation promotes angiogenesis and breast cancer via angiopoietin-like 4. Oncogene, 2019, 38(13):2351-2363. Thus, in certain embodiments, this disclosure contemplates using R-propranolol isomer for the treatment or prevention of breast cancer wherein the subject is obese.

Li et al. report antibody treatment against angiopoietin-like 4 reduces pulmonary edema and injury in secondary pneumococcal pneumonia. mBio, 2019, 10(3): e02469-18. Li et al. Angiopoietin-like 4 Increases Pulmonary Tissue Leakiness and Damage during Influenza Pneumonia, Cell Rep, 2015, 10(5):654-663. Thus, in certain embodiments, this disclosure contemplates using R-propranolol isomer for the treatment or prevention of a bacterial infection, viral infection, or other pathogenic infections, such as pneumococcal pneumonia and influenza, optionally in combination with an antimicrobial or antibiotic agent, e.g., to reduce lung tissue damage.

Jeong et al. report nanoparticles containing SOX duo and ANGPT4 shRNA for osteoarthritis treatment. Thus, in certain embodiments, this disclosure contemplates using R-propranolol isomer for the treatment or prevention of arthritis, psoriatic arthritis, or osteoarthritis.

Tjeerdema et al. report inflammation increases plasma angiopoietin-like protein 4 in patients with the metabolic syndrome and type 2 diabetes. BMJ Open Diabetes Res Care, 2014, 2(1):e000034. Thus, in certain embodiments, this disclosure contemplates using R-propranolol isomer for the treatment or prevention of metabolic syndrome or diabetes, type 1 or type 2 diabetes.

Chakraborty et al. report angiopoietin like-4 as a novel vascular mediator in capillary cerebral amyloid angiopathy. Brain, 2018, 1;141(12):3377-3388. Thus, in certain embodiments, this disclosure contemplates using R-propranolol isomer for the treatment or prevention of dementia

In certain embodiments, the R-propranolol isomer is contemplated to be useful as a general angiogenesis inhibitor with benefits for treating or preventing inflammatory diseases or conditions. In certain embodiments, inflammatory diseases or conditions include inflammatory bowel disease and psoriatic arthritis. In certain embodiments, inflammatory diseases or conditions include multiple sclerosis, lupus, and hidradenitis suppurativa.

In certain embodiments, the R-propranolol isomer is contemplated to be useful for treating or preventing auto immune diseases or conditions. In certain embodiments, the R-propranolol isomer is contemplated to be useful for treating or preventing achalasia, Addison's disease, adult Still's disease, agammaglobulinemia, alopecia areata, amyloidosis, ankylosing spondylitis, anti-GBM/Anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, castleman disease (CD), celiac disease, chagas disease. chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammaglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud' s phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), Takayasu' s arteritis, temporal arteritis/giant cell arteritis, thrombocytopenic purpura (TTP), Tolosa-Hunt syndrome (THS), transverse myelitis, ulcerative colitis (UC), undifferentiated connective tissue disease (UCTD), uveitis, vasculitis, vitiligo, or Vogt-Koyanagi-Harada Disease.

Yang et al. report ANGPTL4 has implications in a number of diseases and conditions. Bioscience Reports, 2018, 38 BSR20180557. In certain embodiments, the R-propranolol isomer is contemplated to be useful for treating or preventing ischemic retinopathies such as diabetic retinopathy (DR), age-related macular degeneration (AMD), retinal vein occlusion, and sickle cell retinopathy (SCR). In certain embodiments, the R-propranolol isomer is contemplated to be useful for treating or preventing hypertriglyceridemia and hepatic steatosis. In certain embodiments, the R-propranolol isomer is contemplated to be useful for treating or preventing metabolic syndrome, cardiovascular events, heart attack, ischemic heart disease, dyslipidemia and coronary artery disease (CAD).

In certain embodiments, the R-propranolol isomer is contemplated to be useful for treating or preventing proteinuria and hypertriglyceridemia in nephrotic syndrome. In certain embodiments, the R-propranolol isomer is contemplated to be useful for treating or preventing osteoarthritis. In certain embodiments, the R-propranolol isomer is contemplated to be useful for treating or preventing cartilage destruction. In certain embodiments, the R-propranolol isomer is contemplated to be useful for treating or preventing diabetic eye disease. In certain embodiments, the R-propranolol isomer is contemplated to be useful for treating or preventing branch retinal vein occlusion. In certain embodiments, the R-propranolol isomer is contemplated to be useful for treating or preventing branch retinal artery occlusion. In certain embodiments, the R-propranolol isomer is contemplated to be useful for treating or preventing pterygium. In certain embodiments, the R-propranolol isomer is contemplated to be useful for treating or preventing uveal melanoma. In certain embodiments, the R-propranolol isomer is contemplated to be useful for treating or preventing retina edema.

Enantiomerically enriched pharmaceutical products comprising R-propranolol are contemplated for all uses disclosed herein; however, for any of the methods disclosed herein, it is also contemplated that a racemic mixture comprising the R-propranolol isomer can also be used.

### Pharmaceutical Compositions

Pharmaceutical compositions typically comprise an effective amount of compounds and a suitable pharmaceutical acceptable carrier. The preparations can be prepared in a manner known per se, which usually involves mixing the compounds according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

The composition comprising (R)-(+)-1-(isopropylamino)-3-(naphthalen-1-yloxy)propan-2-ol or salt thereof in enantiomeric excess as reported herein s of the present disclosure can be administered to a subject either alone or as a part of a pharmaceutical composition. In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof.

The pharmaceutical compositions of the present disclosure can be administered to subjects either topically to the skin, orally, rectally, parenterally (intravenously, intramuscularly, or subcutaneously), intracisternally, intravaginally, intraperitoneally, intravesically, locally (powders, ointments, or drops), or as a buccal or nasal spray. Pharmaceutically acceptable salts, solvates and hydrates of the compounds listed are also useful in the method of the disclosure and in pharmaceutical compositions of the disclosure.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain adjuvants such as preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar and as high molecular weight polyethylene glycols, and the like.

Solid dosage forms such as tablets, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Controlled slow release formulations are also preferred, including osmotic pumps and layered delivery systems.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions may contain suspending agents, as for example, ethoxylated iso-stearyl alcohols, polyoxyethylene sorbitol and sorbitan esters, microcrystalline cellulose, aluminum hydroxide, bentonite agar-agar and tragacanth, or mixtures of these substances, and the like.

Compositions for rectal administrations are preferably suppositories that can be prepared by mixing the compounds of the present disclosure with suitable nonirritating excipients or carriers such as cocoa butter, polyethylene glycol or a suppository wax, which are solid at ordinary temperatures but liquid at body temperature and therefore, melt in the rectum or vaginal cavity and release the active component.

Dosage forms for topical administration of a composition comprising (R)-(+)-1-(isopropylamino)-3-(naphthalen-1-yloxy)propan-2-ol or salt thereof in enantiomeric excess of this disclosure include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this disclosure.

Pharmaceutical compositions typically comprise an effective amount of a composition comprising (R)-(+)-1-(isopropylamino)-3-(naphthalen-1-yloxy)propan-2-ol or salt thereof in enantiomeric excess and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet, 2006, 21(3):173-85, entitled “Human carboxylesterase isozymes: catalytic properties and rational drug design.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

### EXAMPLES

### R(+)-propranolol is More Effective in Downregulating Angptl4 than Beta Blocker Active S-(−)-propranolol

Genetic studies of hemangiomas have not elucidated a common genetic cause, as opposed to other vascular lesions, such as vascular malformations and angiosarcomas, in which genetic mutations have been found. Cytokines have been found in hemangiomas of infancy, including vascular endothelial growth factor (VEGF) and Angiopoietin-2 (Ang-2). The role of these factors in hemangiomas are not fully understood, especially given that Ang-2 can serve as a growth factor or mediate endothelial apoptosis depending on the context. A series of infants were studied who received propranolol for treatment of hemangioma of infancy. It was found that propranolol reduced VEGF levels in saliva but not Ang-2. This suggests that propranolol is a) not acting to decrease Ang-2, and b) other angiogenic cytokines might be playing a role.

Human endothelial stem cells derived from hemangiomas of infancy were treated with R and S propranolol, as well as gentian violet, an angiogenesis inhibitor that is used on ulcerated hemangiomas. One of the genes that was downregulated by all treatments was Angptl4. Interestingly, the inactive R-propranolol was far more effective in downregulating Angptl4 than beta-blocker active S-propranolol. Angptl4 is present in authentic hemangiomas of infancy. R-propranolol is active in vivo against bend3 cells, a murine model of hemangioma of infancy. Thus, it is contemplated that beta blockade is not required for the effect of propranolol for hemangiomas of infancy. Thus, infants could be treated with propranolol and avoid the side effects of beta blockade. Potentially higher doses of R-propranolol could be administered to treat hemangiomas and other angiogenic disorders.

### R-propranolol Treatment Significantly Reduces the Xenograft Tumor Volume

The xenograft model was developed and approved by the Institutional Animal Care and Use Committee of Emory University. bEnd.3 cell suspension in growth medium was inoculated in the right flank of athymic Nu/Nu nude male mice purchased from the Charles River Laboratories at 2.5×10⁵ cells/mouse (n=5 per group). Control or R-propranolol was administered intraperitoneally five times a week at 3.5 mg/kg/day. R-propranolol treatment was initiated on the second day after the tumor cell injection.

To elucidate the signaling cascade independent of beta blockade in hemangioma formation, tie-2-deficient mouse hemangioma cell line, bEnd.3 was xenografted and treated with or without R-propranolol, isomer deficient in beta blockade activity. R-propranolol group showed significant tumor volume reduction from 593.32±mm3 to 37.60±mm3 (p=0.014) as shown in FIG. 3.

### Gene Array and Western Analysis of HemSc Cells

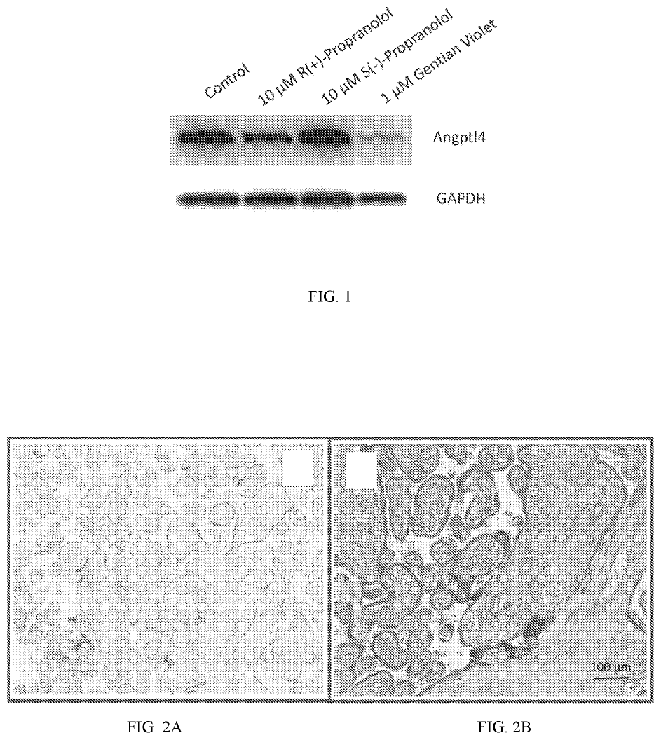

HemSC cells were treated with R and S-propranolol, gentian violet, and vehicle control. RNA was harvested and subject to gene array, with a particular focus of genes coordinately regulated by all three treatments compared to vehicle control. The most regulated gene by all three treatments was Angptl4. HemSC was treated with each isoform of propranolol and gentian violet, and Western blot analysis of Angptl4 was performed. Surprisingly, the R-propranolol, not the beta blocker isomer S-propranolol, reduced expression of Angptl4. Gentian violet also downregulated Angptl4 (FIG. 1).

### RNAseq Analysis of R-Propranolol vs Vehicle Treated Tumors

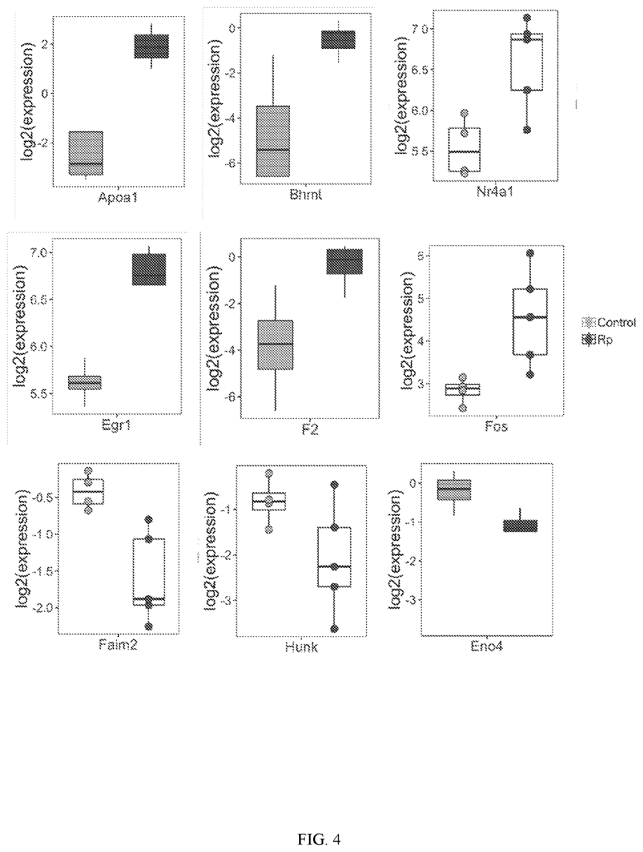

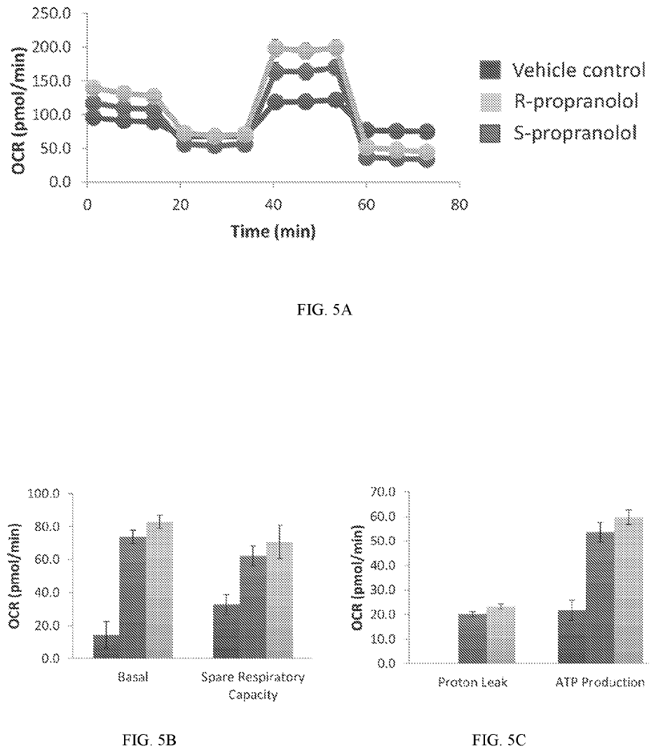

In order to gain an understanding of the mechanism of action of R-propranolol in vivo, vehicle and R-propranolol treated tumors were subjected to RNA seq analysis. KEGG analysis showed that choline metabolism was the pathway affected by R-propranolol treatment. Consistent with this, the most highly regulated gene by R-propranolol treatment was BHMT. Egr-1, an AP-1 subunit with tumor suppressive activity, was also elevated by R-propranolol treatment. APOA1 was also induced by R-propranolol. (FIG. 4). The APOA1 gene expresses a protein called apolipoprotein A-I (apoA-I). ApoA-I is a component of high-density lipoprotein (HDL) which transports cholesterol and phospholipids through the bloodstream for excretion by the liver beneficial for cardiovascular health.

### Immunohistochemistry of Human Hemangioma of Infancy for Angptl4

In order to demonstrate physiologic relevance, experiments were performed to determine whether Angptl4 is expressed in human hemangioma of infancy in situ. Immunohistochemistry demonstrates expression of Angptl4. A negative control was used without antibody (FIG. 2).

What is claimed is:

1. A pharmaceutical composition comprising (R)-(+)-1-(isopropylamino)-3-(naphthalen-1-yloxy)propan-2-ol or salt thereof in enantiomeric excess and a pharmaceutically acceptable excipient.

2. The pharmaceutical composition of claim 1, wherein the enantiomeric excess is 98% or greater.

3. The pharmaceutical composition of claim 1 in the form of a tablet, pill, capsule, gel, gel capsule or cream.

4. The pharmaceutical composition of claim 1 in the form of a sterilized pH buffered aqueous salt solution or a saline phosphate buffer between a pH of 6 to 8, optionally comprising a saccharide or polysaccharide.

5. The pharmaceutical composition of claim 1 in solid form surrounded by an enteric coating.

6. The pharmaceutical composition of claim 5, wherein the enteric coatings comprises methyl acrylate-methacrylic acid copolymers, cellulose acetate phthalate (CAP), cellulose acetate succinate, hypromellose (hydroxypropyl methylcellulose), hypromellose phthalate (hydroxypropyl methyl cellulose phthalate), hypromellose acetate succinate (hydroxypropyl methyl cellulose acetate succinate), diethyl phthalate, polyvinyl acetate phthalate (PVAP), methyl methacrylate-methacrylic acid copolymers, or combinations thereof

7. The pharmaceutical composition of claim 1, wherein the pharmaceutically acceptable excipient is selected from lactose, sucrose, mannitol, triethyl citrate, dextrose, cellulose, microcrystalline cellulose, methyl cellulose, ethyl cellulose, hydroxyl propyl cellulose, hydroxypropyl methylcellulose, carboxymethylcellulose, croscarmellose sodium, polyvinyl N-pyrrolidone (crospovidone), ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

8. A method of treating or preventing angiogenic disorders of the skin comprising administering an effective amount of a composition of claim 1 to a subject in need thereof.

9. A method of treating or preventing hemangiomas comprising administering an effective amount of a composition of claim 1 to a subject in need thereof.

10. A method of treating or preventing skin cancer comprising administering an effective amount of a composition of claim 1 to a subject in need thereof.

11. A method of treating or preventing melanoma comprising administering an effective amount of a composition of claim 1 to a subject in need thereof.

12. The method of claim 8, wherein the composition is administered in combination with another chemotherapy agent, beta-blocker, or combinations thereof.

13. The method of claim 12, wherein the chemotherapy agent is abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, rib ociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

14. The method of claim 12, wherein the beta-blocker is selected from timolol, carvedilol, metoprolol, atenolol, acebutolol, and combinations thereof.

15. A method of treating or preventing atopic dermatitis comprising administering an effective amount of a composition of claim 1 to a subject in need thereof.

16. A method of treating or preventing psoriasis comprising administering an effective amount of a composition of claim 1 to a subject in need thereof.

17. A method of treating or preventing inflammation comprising administering an effective amount of a composition of claim 1 to a subject in need thereof.
